(19) United States
(12) Patent Application Publication
(10) Pub. No.: US 2015/0111226 A1
(43) Pub. Date: Apr. 23, 2015

(54) METHOD FOR PEPTIDE HISTOCHEMICAL DIAGNOSIS

(71) Applicant: National Taiwan University, Taipei City (TW)

(72) Inventors: Chin-Tarng Lin, Taipei City (TW); Han-Chung Wu, Taipei City (TW)

(73) Assignee: National Taiwan University, Taipei City (TW)

(21) Appl. No.: 14/165,358

(22) Filed: Jan. 27, 2014

(30) Foreign Application Priority Data
Oct. 18, 2013 (TW) 102137765

(51) Int. Cl. G01N 33/574

(52) U.S. Cl. CPC G01N 33/57492; G01N 33/57407; G01N 33/57415; G01N 33/57423; G01N 33/57438
USPC 435/7.23

(57) ABSTRACT

The present invention provides a method of peptide histochemical diagnosis to detect the peptide binding protein in the cancer tissue. This peptide binding specifically to tumor cells is linked to the dextran coated iron oxide nanoparticle. The peptide linked dextran coated iron oxide nanoparticle can be used to bind to the formalin-fixed and paraffin-embedded tumor surgical specimens, and the method of present invention can be used to evaluate the efficacy of peptide-targeted chemotherapy for treatment of cancer patients.

### CROSS-REFERENCE TO RELATED APPLICATIONS

This application claims the priority benefit of Taiwan application serial no. 102137765, filed on 18 Oct. 2013. The disclosure of the Taiwan application is incorporated by reference herein.

### BACKGROUND OF THE INVENTION

1. Field of the Invention

The invention relates to a method for peptide histochemical diagnosis, and in particular, to a method for peptide histochemical diagnosis of the formalin-fixed paraffin-embedded surgical specimens.

2. The Prior Arts

Currently, some targeted peptides for the targeted chemotherapy in cancers have been reported. However, it is difficult to identify whether the peptides can be used to bind to the peptide binding protein in cancer cells by staining the formalin-fixed paraffin-embedded surgical specimens, to confirm the peptides for the targeted chemotherapy in cancers has efficacy in patients before clinical use. Although the patients suffer the same type of cancer, it is still unknown that the peptides for the targeted chemotherapy in different patient cancers can really bind to the individual cancer cells of the patients due to the individual difference. Therefore, to identify whether the peptide can bind to the cancer cells of each patient before chemotherapy, it will be extremely beneficial for the efficacy of peptide-targeted chemotherapy.

Thus, it is the key to evaluate therapy effects of the patient before peptide-targeted chemotherapy, and the main problem is to identify whether the targeted peptide can directly bind to the surgical tumor specimens from the patient. Moreover, the peptides have the following characteristics: a. The peptides can bind to different and undifferentiated nasopharyngeal carcinoma (NPC) or other cancer cells; b. The peptides targeted chemotherapy can make tumor shrink; c. The peptides can not bind to the normal cells in the normal organ tissue. It has been reported that biotin can be directly linked to the peptide to form a biotin-peptide, the biotin-peptide can also bind to cancer cells, but it has poor result in the paraffin-embedded surgical specimens. The biotin-peptide can bind to the small surgical specimens a little, and the binding capability is very weak than that of general antibodies, especially, biotin-peptide almost can not bind to the surgical specimens with more than 1 cm. The main reason is that the surgical specimens are formalin-fixed paraffin-embedded surgical sections, and there are only three amino acids of the biotin-peptide can bind to the cancer cells embedded in the surgical sections, the binding ability between the peptide and formalin-fixed paraffin-embedded surgical section is very weak, so almost no binding phenomenon can be observed. It is impossible to anticipate the efficacy of chemotherapy for cancer patient after surgery using this method. Currently, there is not yet a method of pathological diagnosis for targeted peptide to bind to formalin-fixed paraffin-embedded specimens in surgical sections.

### SUMMARY OF THE INVENTION

The present invention provides a method of peptide histochemical diagnosis, which solves the problem to stain the tumor cells of surgical specimens fixed in formalin and embedded in paraffin sections as the relationship between antibody and antigen. In other words, before peptide-targeted chemotherapy applied to the patients with cancer, it needs to concern about the individual differences in tumor cells of the patients clinically. Prior to the targeted peptides chemotherapy, the method of the present invention is used to identify whether the targeted peptides can bind to the tumor cells of the patients, and the targeted peptides chemotherapy can be used to treat the patient if the binding phenomenon is observed.

In the one aspect, the present invention provides for peptide histochemical diagnosis, comprising: a. Providing a paraffin section of formalin-fixed tumor specimen obtained from sectioning and deparaffinzing a tumor specimen embedded in paraffin block; b. Providing a targeted peptide-dextran coated iron oxide nanoparticle, the surface of the iron oxide nanoparticle is coated with dextran, a N-terminal of the targeted peptide is linked to the dextran; and c. Incubating the paraffin section of tumor specimen (after retrieval of binding protein) with the targeted peptide-dextran coated iron oxide nanoparticle, then staining with a reagent to reveal a specific color, wherein a tumor cell in the paraffin section of tumor specimen reveals the specific color, and a normal cell in the paraffin section of tumor specimen is not stained; the reagent is Prussian blue reagent, the specific color of Prussian blue reagent reacting with the targeted peptide-dextran coated iron oxide nanoparticles is blue.

In the present invention, the paraffin section of tumor specimen is embedded in paraffin wax after formalin-fixed and dehydration. In addition, the tumor specimen further processes a high-pressure treatment (for retrieval of binding protein) to obtain the paraffin section of tumor specimen after deparaffinization.

In the present invention, the targeted peptide-dextran coated iron oxide nanoparticles are synthesized by at least 10 targeted peptides linked to dextran coated iron oxide nanoparticle, and the targeted peptide of the targeted peptide-dextran coated iron oxide nanoparticles binds to the tumor cells.

In the present invention, the tumor is nasopharyngeal carcinoma (NPC), breast cancer, hepatoma, pancreatic cancer, small cell lung cancer (SCLC) or neuroblastoma.

In another aspect, the present invention also provides a method of a targeted peptide-dextran coated iron oxide nanoparticle for detecting the peptide binding protein in a paraffin section of tumor specimen, wherein a normal cell in the paraffin section of tumor specimen is not stained, the paraffin section of tumor specimen reveals a color if a tumor cell in the paraffin section of tumor specimen bound by the targeted peptide-dextran coated iron oxide nanoparticle.

Peptide histochemical diagnosis of the present invention, which comprises to use a targeted peptide-dextran coated iron oxide nanoparticle, the N-terminal of the targeted peptide is linked to dextran, and each targeted peptide-dextran coated iron oxide nanoparticle can link to at least 10 targeted peptides; the C-terminal of the targeted peptide of the targeted peptide-dextran coated iron oxide nanoparticle can bind to the tumor cells. Therefore, when the targeted peptide-dextran coated iron oxide nanoparticle of the present invention used to react with paraffin section of tumor specimen, 10 targeted peptides can bind to the tumor cells. The targeted peptide-dextran coated iron oxide nanoparticle of the present invention can reveal blue color after iron oxide nanoparticles reacting with Prussian blue reagent, and the reaction product precipitates in the tumor cells bound by the targeted peptides but not in the cytoplasm of the normal epithelial cells or fibroblasts, and a little reaction products are seen in the membrane of the tumor cells. In summary, the reaction products present in the tumor cells rather than normal cells, although normal cells may show very little of the targeted protein in its cytoplasm (the mononuclear cells may show a little more) but not show in their membranes.

The targeted peptide in the present invention is the short sequence having only 12 amino acids, and the targeted peptides are small molecular compared to the antibody, therefore, after binding to the nanoparticles, the short sequence peptide still have a tiny volume, so the targeted peptide-linked dextran-coated iron oxide nanoparticles can be allowed to enter into the extracellular space. Additionally, one nanoparticle can be linked by many short sequence peptides, which allows more binding ability for targeting tumor cells.

Thus, prior to apply the targeted peptide chemotherapy to treat a cancer patient, peptide histochemical diagnosis of the present invention can be used to identify whether the targeted peptides can bind to a paraffin section of tumor specimen of the patients, and then the targeted peptide chemotherapy can directly be used to treat the patient if the binding phenomenon is observed, which makes therapy effectively.

### BRIEF DESCRIPTION OF THE DRAWINGS

FIGS. 1A and 1B are graphs depicting flow cytometry analysis of FITC (Fluorescein isothiocyanate)-targeted peptide SEQ ID NO: 1 binding to the nasopharyngeal carcinoma (NPC)-TW07 and other cancer cell lines. FIG. 1A shows a strong binding activity of the targeted peptide SEQ ID NO: 1 to NPC cells. The NPC cells incubated with PBS (phosphate buffered saline) are used as a negative control. The FITC-linked control peptide SEQ ID NO: 3 and the biotin-linked targeted peptide SEQ ID NO: 1 or biotin-linked the control peptide all shows very weak binding activity. In FIG. 1B is the flow cytometry analysis of the FITC-targeted peptide SEQ ID NO:1 binding to various cancer cell lines but not immortalized cell line. The picks shown from left to right are human immortalized embryonic renal cells (239T), NPC-TW01, lung cancer line (A549), breast cancer (MB157), neuroblastoma (Be2C), NPC-TW07 and lung cancer (H1299) cell lines. All shows a clear binding peak except the immortalized embryonic renal cell line (293T) without showing any binding phenomenon

FIG. 2A and FIG. 2D are the images of localization of the targeted peptide binding protein in nasopharyngeal carcinoma (NPC) cells by incubation of detergent permealized formalin-fixed NPC cells incubated with biotin linked control peptide (FIG. 2A) and biotin linked targeted peptide (FIG. 2B), and are visualized by Avidin-biotin peroxidase complex and peroxidase substrate diaminobenzidine (DAB). The NPC tumor cells show DAB reaction product in most of tumor cell cytoplasm (FIG. 2D) but not in the control peptide treated cells (FIG. 2A). FIG. 2B-FIG. 2F are the images of localization of the targeted peptide SEQ ID NO: 1 binding protein in NPC cell; the control peptide is SEQ ID NO: 3; FIG. 2B and FIG. 2E are the images of the targeted peptide SEQ ID NO: 1-linked dextran-coated iron oxide nanoparticles (P-Dex-Fe₃O₄) incubated with NPC cell lines and stained with Prussian blue reagent, and the tumor cells reveal Prussian blue reaction products. FIG. 2E is a high magnification of FIG. 2B. FIG. 2C is the image of control peptide-linked dextran-coated iron oxide nanoparticles (CP-Dex-Fe₃O₄) and reveals some non-specific reaction product attached to a few tumor cells. FIG. 2F is a high magnification of FIG. 2C.

FIG. 3A-FIG. 3C are the images of Prussian blue staining for breast cancer cell line (MDA-MB-231) treated with dextran-coated iron oxide nanoparticles (Dex-Fe₃O₄), the targeted peptide SEQ ID NO:1-linked dextran-coated iron oxide nanoparticles (P-Dex-Fe₃O₄) and FITC-targeted peptide SEQ ID NO:1-linked dextran-coated iron oxide nanoparticles (FITC-P-Dex-Fe₃O₄). FIG. 3A is breast cancer cells incubated with Dex-Fe₃O₄, FIG. 3C is cancer cells treated with P-Dex-Fe₃O₄, FIG. 3B and FIG. 3D are cancer cells treated with FITC-P-Dex-Fe₃O₄. FIG. 3A shows no reaction product. FIG. 3B and FIG. 3C show blue reaction product in the cytoplasm of breast cancer cells. FIG. 3D shows the fluorescent signal in the tumor cells as blue reaction product shown in FIG. 3B. Bar in each panel=100 μm.

FIG. 4A-FIG. 4D are the images of Prussian blue staining for blood monocytes, hepatoma (Huh 7) and pancreatic cancer cell line (Pan 1) treated with P-Dex-Fe₃O₄ for 1 hr at 4° C. and 4 hr at 37° C. and fixed for Prussian blue reaction; the targeted sequence is SEQ ID NO: 1. FIG. 4A and FIG. 4B show no blue reaction product in blood monocytes. FIG. 4A is the control panel of FIG. 4C, and FIG. 4B is the control panel of FIG. 4D. FIG. 4A and FIG. 4B all show that P-Dex-Fe₃O₄ do not bind to normal blood cells. FIG. 4C and FIG. 4D show blue reaction product in hepatoma and pancreatic cancer cell lines, respectively.

FIG. 5A-FIG. 5C are the images of Prussian blue staining for hepatoma cell lines (Hep G2) treated with the targeted peptide. In FIG. 5A, the targeted sequence is SEQ ID NO: 1, in FIG. 5C, the targeted sequence is SEQ ID NO: 2. FIG. 5A and FIG. 5C show blue reaction products of the targeted peptide SEQ ID NO:1-linked dextran-coated iron oxide nanoparticles and the targeted peptide SEQ ID NO:2-linked dextran-coated iron oxide nanoparticles binding to hepatoma cell lines (Hep G2) separately; FIG. 5B shows the control iron oxide nanoparticle not binding to hepatoma cell line (Hep G2).

FIG. 6A-FIG. 6F are the images of peptide histochemical localization of peptide targeted protein in NPC biopsy specimens. FIG. 6A and FIG. 6C are the images of NPC biopsy specimens treated with the control peptide (SEQ ID NO: 3-Dex-Fe₃O₄). FIGS. 6A, B, D and E are the images of NPC biopsy specimens counterstained with nuclear fast red. FIG. 6C and FIG. 6F show the images of NPC biopsy specimens without counterstaining with nuclear fast red. FIGS. 6B, D, E and F show the binding results of targeted peptide SEQ ID NO: 1-linked dextran-coated iron oxide nanoparticle (P-Dex-Fe₃O₄) in NPC biopsy specimens from different NPC patients. FIGS. 6B, D and E are the images stained by Prussian blue reagent and counterstained with nuclear fast red. FIG. 6F is the image only stained by Prussian blue reagent. In tumor nests, there are Prussian blue reaction products in most NPC biopsy specimens (as shown in 6B, D, E and F). The FIGS. 6B, D and E show clear staining of infiltrating tumor cells in stromal regions. Bar in each panel=25 μm.

FIG. 7A-FIG. 7F are the images of peptide histochemical localization of peptide targeted protein in breast cancer surgical specimens from different patients. FIGS. 7B-F and FIGS. 7H-L are the images of breast cancer surgical specimens treated with the targeted peptide SEQ ID NO:1-linked dextran-coated iron oxide nanoparticles (P-Dex-Fe₃O₄). FIG. 7A and FIG. 7G are the images of breast cancer surgical specimens treated with control peptide-linked dextran-coated iron oxide nanoparticle (CP-Dex-Fe₃O₄) showing no Prussian blue reaction product. FIGS. 7A-F are the images stained by Prussian blue reagent and counterstained with nuclear fast red, FIGS. 7G-L are the images only stained by Prussian blue reagent. FIGS. 7B-F shows more or less Prussian blue reaction product in tumor cells in each breast cancer surgical section. In the stromal region, there are no reaction product in endothelial cells and fibroblasts. FIG. 7K and FIG. 7L show the results of the stained breast cancer cells in the metastatic axillary lymph nodes, and the blue reaction products only reveal in the tumor cells, but not in the macrophages. In FIG. 7L, there are many carbon-laden macrophages showing no blue reaction products. Bar in FIGS. 7A-J=25 μm; bar in FIG. 7K=100 μm.

FIG. 8A-FIG. 8D are the images of peptide histochemical localization of peptide binding antigen in hepatoma surgical specimens. The surgical specimens are treated with the targeted peptide SEQ ID NO: 1-linked dextran-coated iron oxide nanoparticle (P-Dex-Fe₃O₄) (FIG. 8A and FIG. 8B) and the targeted peptide SEQ ID NO: 2-linked dextran-coated iron oxide nanoparticle, respectively (FIG. 8C). In the right part of FIG. 8A, the normal cells are compressed by tumor cells, and the blue reaction products do not show in the normal cells, but show in single infiltrating tumor cells. FIG. 8B shows a high magnification of hepatoma cells stained by reaction product. FIG. 8C is the image of staining hepatoma surgical specimens treated with the targeted peptide SEQ ID NO: 2-linked dextran-coated iron oxide nanoparticles. FIG. 8D is the image of staining hepatoma surgical specimens treated with the control panel (CP-Dex-Fe₃O₄); no reaction product can be seen.

FIG. 9A-FIG. 9D are the images of peptide histochemical localization of peptide binding protein in pancreatic surgical specimens: pancreatic surgical specimens are treated with the targeted peptide SEQ ID NO:1-linked dextran-coated iron oxide nanoparticles (P-Dex-Fe₃O₄) (FIG. 9A and FIG. 9B) and the targeted peptide SEQ ID NO:2-linked dextran-coated iron oxide nanoparticles (FIG. 9C). FIGS. 9A-C all show blue reaction products. FIG. 9D is the image of staining pancreatic surgical specimens treated with the control peptide (CP-Dex-Fe₃O₄), no reaction product can be seen.

### DETAILED DESCRIPTION OF THE PREFERRED EMBODIMENT

### Definition

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention.

Unless otherwise defined, all technical and scientific terms used herein have the same meaning as commonly understood by one of ordinary skill in the art to which this invention pertains. In the case of conflict, the present document, including definitions will be explained clearly.

As used herein, “targeted peptide” shall generally mean a peptide bind to a cancer cell but not bind to a normal cell.

As used herein, “approximately” shall generally mean within 20 percent, preferably within 10 percent, and more preferably within 5 percent of a given value or range. Numerical quantities given herein are approximate, meaning that the term “approximately” can be inferred if not expressly stated.

### Preparation of Cell Lines, Biopsy and Surgical Specimens and Peptides

Cell Lines:

Cell lines used here include nasopharyngeal carcinoma (NPC-TW01, NPC-TW06 and NPC-TW07), breast cancer (MDA-MB231 and MB157), lung cancer (H1299 and A549), neuroblastoma (Be2C), hepatoma (Hep G2), pancreatic cancer (Pan 1) cell lines and immortalized embryonic renal epithelia (TEKID) (293T): all cell lines were cultured in the DMEM containing L-glutamine and 10% fecal calf serum and incubated in the 10% CO₂ incubator as routine cell culture condition.

Biopsy and Surgical Specimens:

The biopsy specimens and surgical specimens of nasopharyngeal carcinoma, breast cancer, hepatoma and pancreatic cancer are obtained from the archives of the Department of Pathology at the National Taiwan University Hospital (NTUH) with the approval for usage by NTUH Institution Review Board (IRB).

Peptide:

Peptides include: (1) targeted peptide SEQ ID NO:1 and SEQ ID NO:2; (2) control peptide SEQ ID NO:3; (3) FITC (Fluorescein isothiocyanate)-targeted peptide SEQ ID NO:1; (4) FITC-control peptide; (5) biotin-targeted peptide SEQ ID NO:1 (B-P); (6) biotin-control peptide (B-C-P); (7) biotin-5 amino acids spacer-targeted peptide SEQ ID NO:1, i.e. biotin-modified-peptide (B-m-P), wherein the sequence of 5 amino acids spacer is SEQ ID NO:4; (8) targeted peptide SEQ ID NO:1-linked dextran-coated iron oxide nanoparticles (P-Dex-Fe₃O₄); and (9) control peptide-linked dextran-coated iron oxide nanoparticles (CP-Dex-Fe₃O₄). Most of the peptides are synthesized by GeneDiveX, Inc, (Las Vegas, U.S.A.), and Dex-Fe₃O₄ is purchased from a commercial source (MagQu Co., Ltd., Taipei, Taiwan).

### Example 1

### Synthesis and Characterization of Magnetic Nanoparticles

The dextran coated-Fe₃O₄ is linked with the targeted peptide SEQ ID NO:1 or the FITC-targeted peptide SEQ ID NO:1 by the MagQu Company. Dextran is used as the hydrophilic surfactant layer. The iron oxide fluid with the desired concentration is available by diluting the highly concentrated iron oxide fluid with a pH 7.4 phosphate buffered saline (PBS) solution. For iron oxide labeling, the NPC-TW01 cells are cultured for 24 hr, incubated with or without the targeted peptide SEQ ID NO:1-linked dextran-coated iron oxide nanoparticle (P-Dex-Fe₃O₄) at a concentration of 10 μg Fe₃O₄/mL in the incubation media for 1 hr at 4° C. and incubated for 4 hr at 37° C. in a 10% CO₂ incubator. Cells are washed in a 2% fetal bovine serum in PBS. After fixation, the binding ability between the targeted peptide SEQ ID NO: 1 and tumor cells can be observed by Prussian blue reagent; each iron oxide nanoparticle is bound by approximately above 10 targeted peptides.

### Example 2

### Flow Cytometry Analysis of the Targeted Peptide SEQ ID NO: 1 Binding Cells in Different Cell Lines

The cultured cells, including nasopharyngeal carcinoma (NPC-TW01 and NPC-TW07), lung cancer (H1299 and A549), neuroblastoma (Be2C), breast cancer (MB157) and immortalized embryonic renal epithelia (TEKID) (239T) are subjected to flow cytometer (FACSCAN, BD Co., U.S.A.) after the FITC (Fluorescein isothiocyanate)-targeted peptide SEQ ID NO:1 treatment. Simultaneously, FITC-control peptide, FITC-biotin-targeted peptide SEQ ID NO: 1 (B-P) and FITC-biotin-control peptide (B-C-P) are used for flow cytometry comparison.

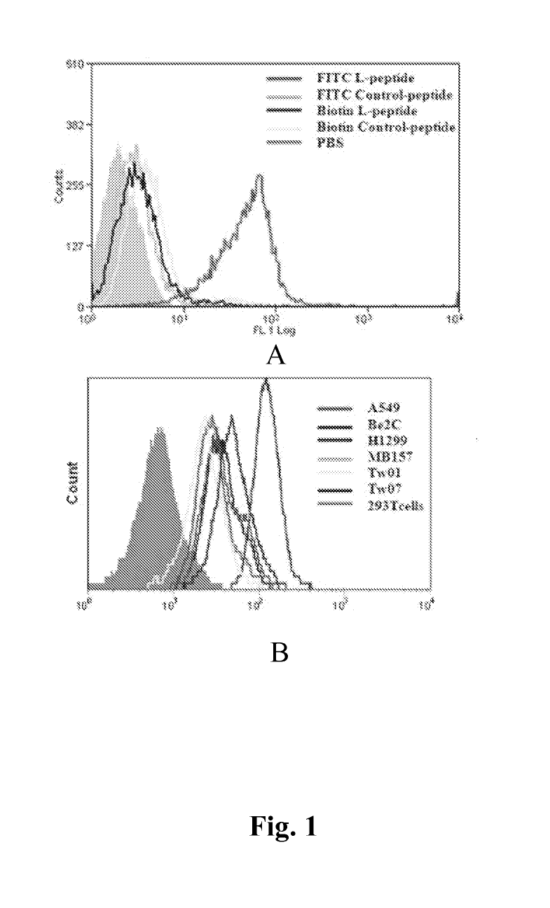

When NPC-TW07 cells are incubated with FITC-targeted peptide SEQ ID NO: 1, a clear peak is found in the histogram (FIG. 1A). When the targeted peptide SEQ ID NO: 1 is linked with biotin, very weak or no binding is seen. When other NPC cell line, such as NPC-TW01, and other cancer lines including lung cancer (A549 and H1299), neuroblastoma (Be2C) and breast cancer (MB157) are incubated with the FITC-targeted peptide SEQ ID NO:1, as shown in FIG. 1B, all shows a clear binding peak except the immortalized renal cell line (293T) without showing any binding phenomenon.

### Example 3

### Localization of the Targeted Peptide SEQ ID NO: 1 in Nasopharyngeal Carcinoma

Because the targeted peptide SEQ ID NO:1 binds weakly to the cancer cells after conjugating to biotin, the sequence of this peptide was modified as Biotin-modified-peptide (B-m-P). B-m-P is modified by adding 5 amino acids spacer SEQ ID NO: 4 to N-terminal and linking with biotin. B-m-P can bind to the small surgical NPC specimens, but the result is not good enough to observe a clear reaction product. For the surgical specimens with more than 1 cm, B-m-P still can not bind to the surgical specimens, there is no binding phenomenon can be observed, while the targeted peptide SEQ ID NO:1-linked dextran-coated iron oxide nanoparticles (P-Dex-Fe₃O₄) of the present invention can bind to tumor cells and be stained with Prussian blue reagent. The result is excellent so as to name “peptide histochemical diagnosis”.

In the present invention, P-Dex-Fe₃O₄ has to be prepared at first. The nanoparticle (MW 60,000 to 70,000) is composed of an iron oxide core coated with dextran polymer. Each dextran-coated iron oxide nanoparticles can be linked with more than 10 targeted peptides.

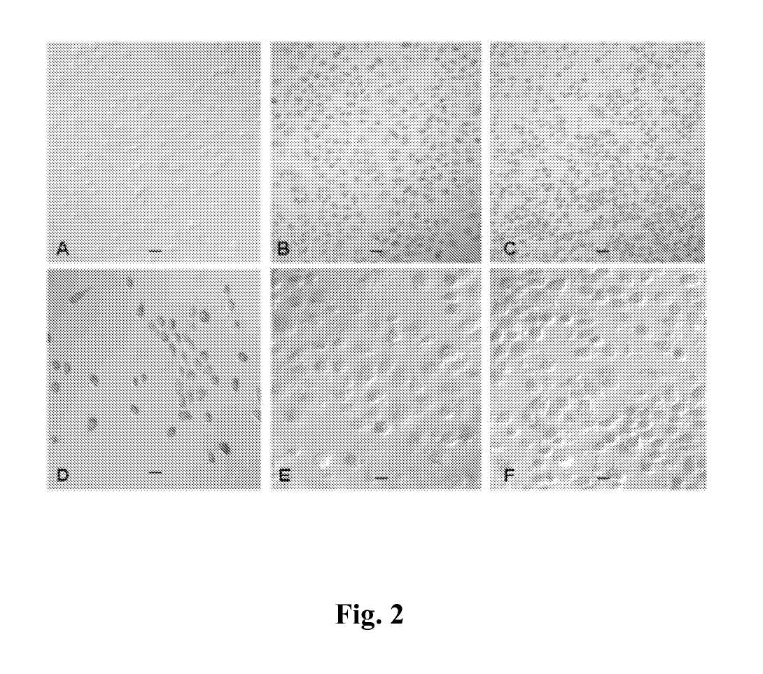

FIG. 2 shows the results of targeted peptide SEQ ID NO:1 binding to NPC cells (NPC-TW01): when the NPC cultured cells are also incubated with P-Dex-Fe₃O₄ nanoparticles at 4° C. for 1.0 hr and 37° C. for 4 hr, and fixed for reacting with Prussian blue reagent, the reaction products are seen in the cytoplasm of a majority of the tumor cells (FIG. 2B and FIG. 2E); however, the tumor cells treated with CP-Dex-Fe₃O₄ reveals no specific reaction product (FIG. 2C and FIG. 2F). If the tumor cells are fixed and permeabilized by protease at first, then incubated with P-Dex-Fe₃O₄, and subjected to avidin-biotin-peroxidase complex and peroxidase treatment, the brown reaction product can be seen in most of the tumor cells (FIG. 2A) but not in the control peptide treated case (FIG. 2A).

### Example 4

### Localization of the Targeted Peptide SEQ ID NO: 1 in Breast Cancer

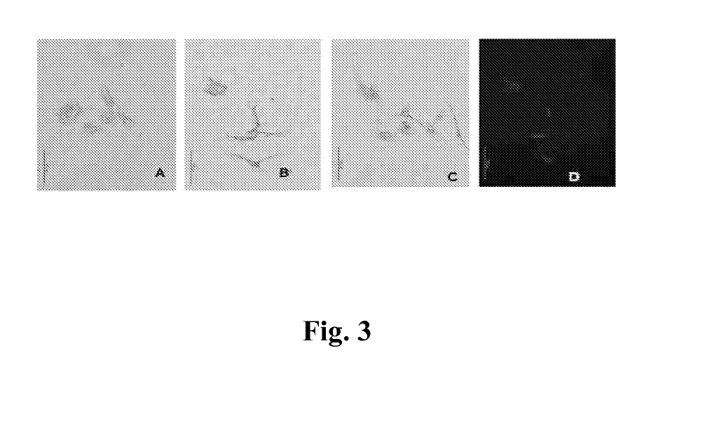

FIG. 3 shows the results of targeted peptide SEQ ID NO:1 binding to breast cancer cells (MDA-MB-231): P-Dex-Fe₃O₄ of the present invention and FITC (Fluorescein isothiocyanate)-targeted peptide SEQ ID NO:1-linked dextran-coated iron oxide nanoparticle (FITC-P-Dex-Fe₃O₄) treated breast cancer cells, respectively, results show that the reaction products appear in most of the cytoplasm of breast cancer cells (FIG. 3C), while no reaction product is seen in the control cells treated with Dex-Fe₃O₄ (FIG. 3A). In FITC-L-P-Dex-Fe₃O₄ nanoparticles treated cells, reaction products are seen in the cytoplasm of breast cancer cells (FIG. 3B), and the fluorescent signal is also seen in the same tumor cells (FIG. 3D).

### Example 5

### Localization of the Targeted Peptide SEQ ID NO: 1 in Blood Monocytes, Hepatoma and Pancreatic Cancer Cells

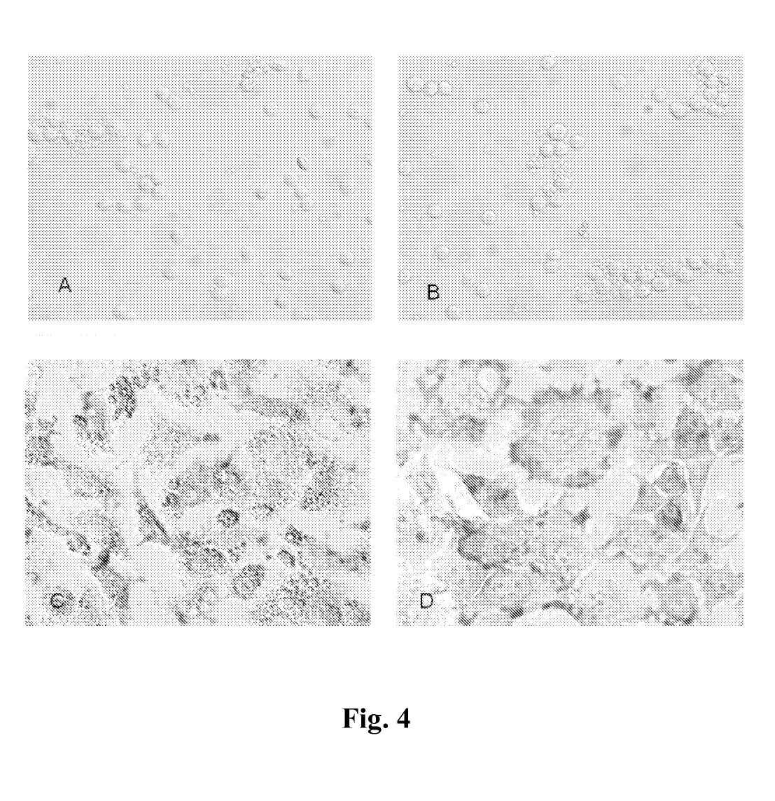

Blood monocytes, hepatoma (Hep G2) and pancreatic cancer (Pan 1) cell lines are incubated with P-Dex-Fe₃O₄ nanoparticle for 1.0 hr and 37° C. for 4 hr, and then are stained with Prussian blue reagent. As shown in FIG. 4C and FIG. 4 D, hepatoma (Hep G2) and pancreatic cancer (Pan 1) cell lines show blue reaction products. But blood monocytes show no reaction product (FIG. 4A and FIG. 4B); these results indicate that the protein bound by the targeted peptide SEQ ID NO: 1 is expressed in cancer cells but not in blood monocytes.

### Example 6

### Localization of the Targeted Peptide SEQ ID NO: 2 in Hepatoma Cells

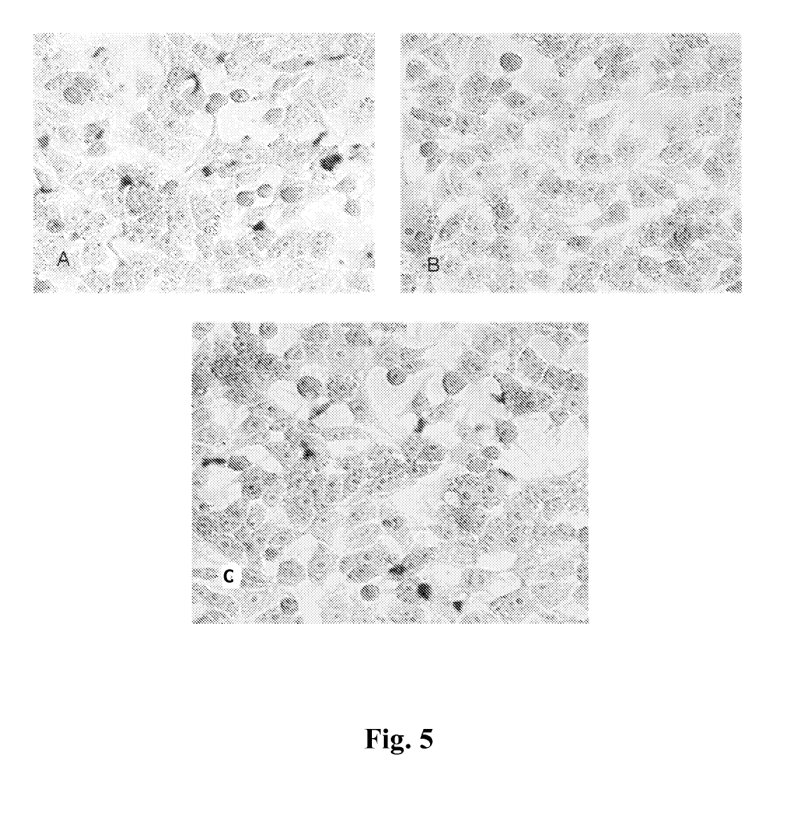

In addition to the targeted peptide SEQ ID NO: 1, the present invention also provides another targeted peptide SEQ ID NO: 2 for synthesizing the targeted peptide SEQ ID NO: 2-linked dextran-coated iron oxide nanoparticles. Hepatoma cell lines (Hep G2) are incubated with the targeted peptide SEQ ID NO: 1-linked dextran-coated iron oxide nanoparticles and the targeted peptide SEQ ID NO: 2-linked dextran-coated iron oxide nanoparticle, respectively, then are reacted with Prussian blue reagent. As shown in FIG. 5A and FIG. 5C, the targeted peptide SEQ ID NO:1-linked dextran-coated iron oxide nanoparticles and the targeted peptide SEQ ID NO:2-linked dextran-coated iron oxide nanoparticles all show blue reaction product, the hepatoma cell lines treated with control peptide-linked dextran-coated iron oxide nanoparticle (CP-Dex-Fe₃O₄) reveals no specific reaction product (FIG. 5B).

In view of abovementioned examples, as long as a targeted peptide can be used to bind to a type of cancer cells, the targeted peptide can be used to bind to cancer cells in surgical paraffin section of the same cancers by using the method of the present invention.

### Example 7

### Localization of the Targeted Peptide SEQ ID NO: 1 in Nasopharyngeal Carcinoma (NPC) Biopsy Specimens

The NPC biopsy specimens are fixed in formalin and embedded in paraffin block then are cut into thin sections with 5 mm After deparaffinizaion, the paraffin-embedded sections are retrieved the binding capability of targeted peptide binding protein with Trilogy (Cell Marque, Rocklin, Calif.), then autoclaved to 132° C.-140° C. for 5-10 min and cooled to room temperature. The sections are incubated overnight at 4° C. with the P-Dex-Fe₃O₄ nanoparticles, and washed with PBS, then are incubated for 15-30 min with routine Prussian blue reagent (containing 2% potassium ferricyanide (Sigma-aldrich.com. St Louis Mo., U.S.A.) and 0.5 N HCL in dH₂O=1:1), followed by counterstaining for 5 min with nuclear fast red solution, washed with water, and mounted with 50% glycerol directly or with balsam after alcohol dehydration.

In the present invention, peptide histochemical diagnosis can be applied not only to cancer cell lines but also to paraffin-embedded cancer sections fixed in formalin. Therefore the peptide histochemical diagnosis of the present invention can be applied to any cancer type section.

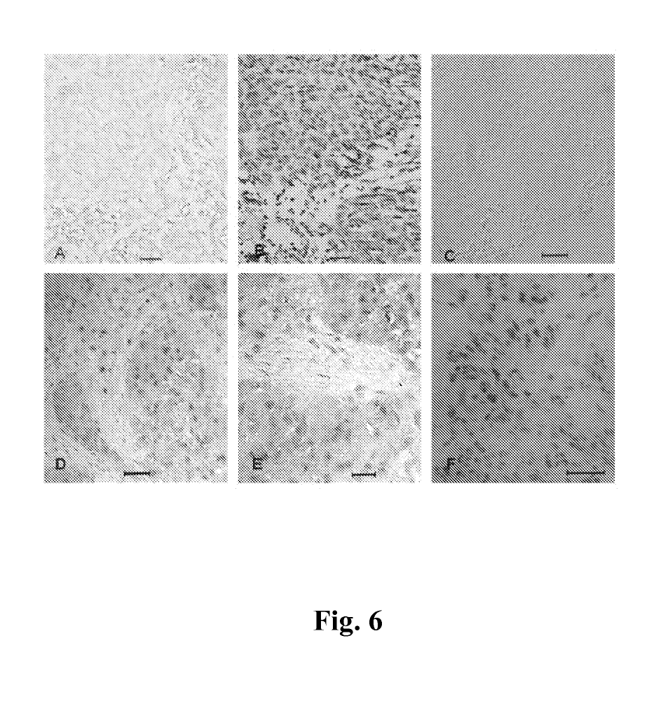

The results show binding capability of the targeted peptide SEQ ID NO: 1 in NPC biopsy specimens. Because the Biotin-modified-peptide (B-m-P) does not bind easily to the formalin-fixed paraffin-embedded tissue section, the present invention provides a method for peptide histochemical diagnosis, in one embodiment, which uses targeted peptide SEQ ID NO: 1-linked dextran-coated iron oxide nanoparticle (P-Dex-Fe₃O₄) binding to NPC cells. The results show that the Prussian blue reaction product is easily identified in the tumor cells of stained sections (FIGS. 6B, D, E, and F). Some tumor cells having infiltrated stromal region also reveal reaction products (FIGS. 6B, D and E). The tumor cells in the tumor nests of NPC biopsy specimens reveal positive reaction products; however, the tumor cells treated with CP-Dex-Fe₃O₄ show no staining (FIG. 6A and FIG. 6C). FIG. 6A is counterstained with nuclear fast red; FIG. 6 C and FIG. 6F are without counterstaining with nuclear fast red.

### Example 8

### Localization of the Targeted Peptide SEQ ID NO: 1 in Breast Cancer Surgical Specimens

In the present invention, peptide histochemical diagnosis can be applied to formalin-fixed paraffin-embedded breast cancer sections.

The results show binding capability of the targeted peptide SEQ ID NO: 1 in breast surgical specimens. When biotin-modified-peptide (B-m-P) is used to bind to the formalin-fixed paraffin-embedded tissue sections, a very weak or no binding signal can be obtained after staining (figure no show). The result indicates that the biotin linked modified targeted peptide SEQ ID NO: 1 (B-m-P) can not easily bind to the formalin-fixed paraffin-embedded tissue sections.

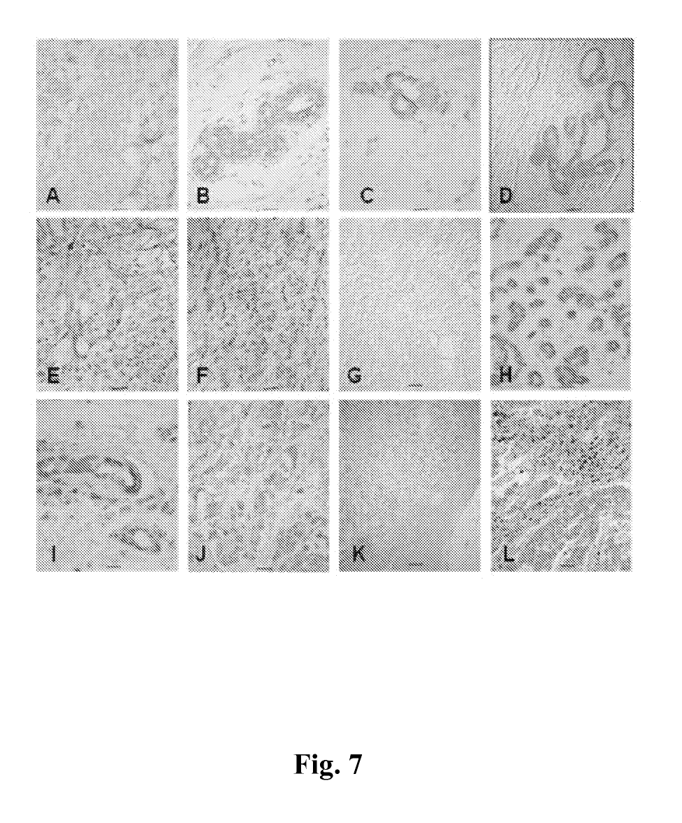

Thus, peptide histochemical diagnosis of the present invention can be applied to localization of the targeted peptide in the formalin-fixed paraffin-embedded breast cancer sections of large surgical specimens with more than 1 cm. After incubation with the targeted peptide SEQ ID NO: 1-linked dextran-coated iron oxide nanoparticle (P-Dex-Fe₃O₄), the formalin-fixed paraffin-embedded breast cancer sections show the Prussian blue reaction products (FIGS. 7 B-F and FIGS. 7H-J), while, the stromal regions in tumor nests reveal no blue reaction product. As shown in FIG. 7K and FIG. 7L, the tumor cells obtained from breast cancer metastatic axillary lymph nodes reveal blue reaction product. However, the breast tumor cells treated with CP-Dex-Fe₃O₄ show no staining (FIG. 7A and FIG. 7G). In short, in breast cancer surgical specimens, the tumor cell treated with the method of present invention reveals positive reaction product.

### Example 9

### Localization of the Targeted Peptide SEQ ID NO: 1 in Hepatoma Surgical Specimens

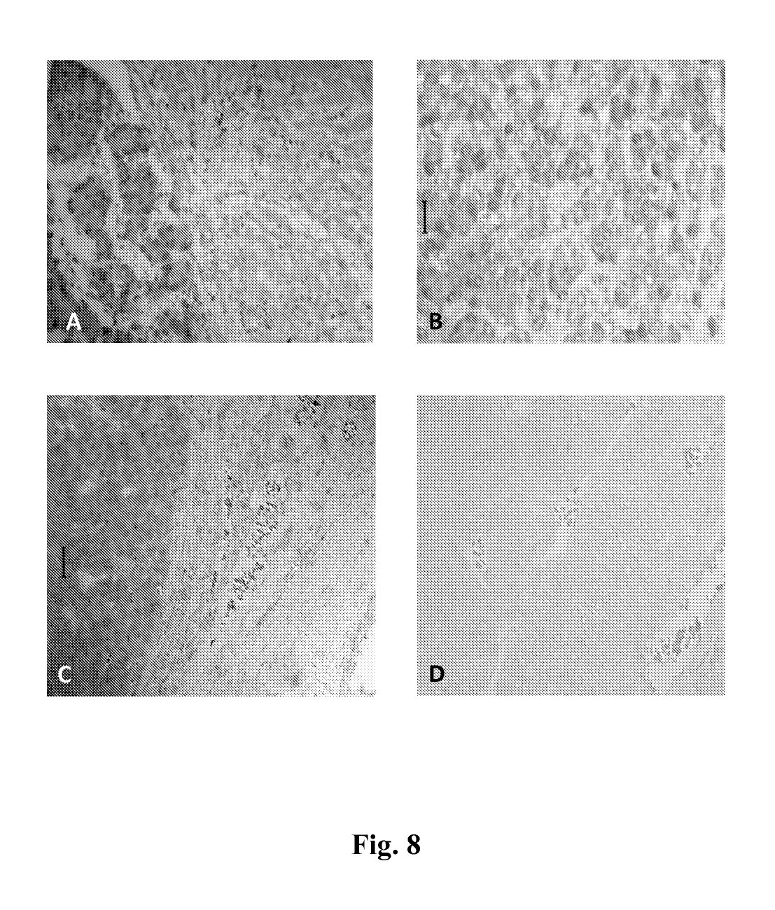

Peptide histochemical diagnosis of the present invention can be applied to hepatoma surgical specimens. Using peptide histochemical technique, the targeted peptide SEQ ID NO: 1-linked dextran-coated iron oxide nanoparticles (P-Dex-Fe₃O₄) are used to bind formalin-fixed paraffin-embedded hepatoma sections (FIG. 8A and FIG. 8B). FIG. 8C shows reaction products of the targeted peptide SEQ ID NO: 2-linked dextran-coated iron oxide nanoparticles binding to hepatoma sections and the Prussian blue products can also be observed in the tumor cells. FIG. 8D is hepatoma cells treated with CP-Dex-Fe₃O₄ and shows no reaction products.

### Example 10

### Localization of the Targeted Peptide SEQ ID NO: 1 and 2 in Pancreatic Surgical Specimens

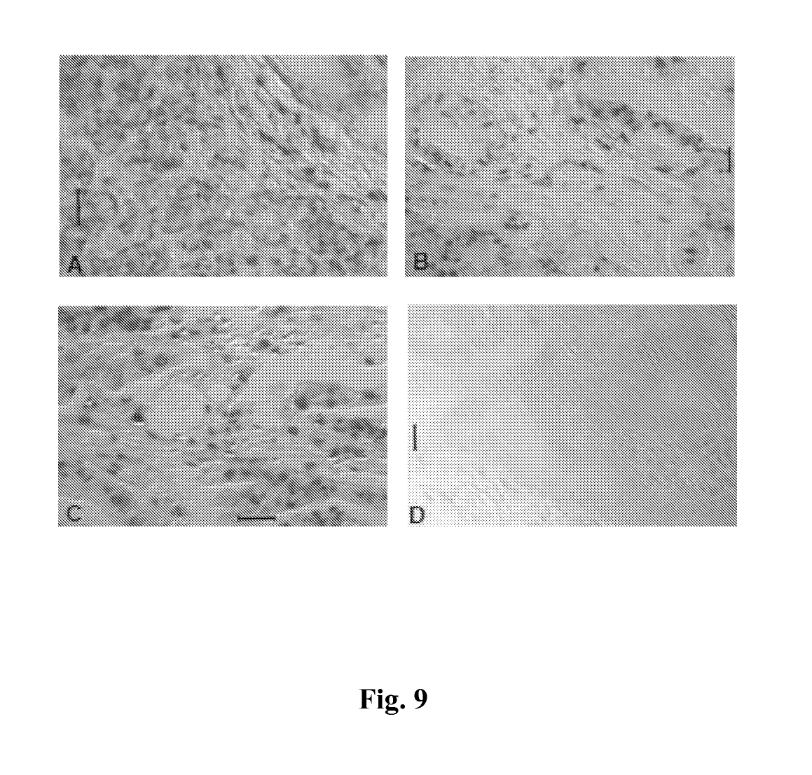

Peptide histochemical diagnosis of the present invention can be applied to pancreatic surgical specimens, ether the targeted peptide is SEQ ID NO: 1 or SEQ ID NO: 2. Using peptide histochemical method, the targeted peptide SEQ ID NO:1-linked dextran-coated iron oxide nanoparticles (P-Dex-Fe₃O₄) (FIG. 9A and FIG. 9C) and the targeted peptide SEQ ID NO:2-linked dextran-coated iron oxide nanoparticles (FIG. 9B) are used to bind formalin-fixed paraffin-embedded pancreatic cancer sections, respectively, and stained with Prussian blue reagent. FIGS. 9A, B and C all shows the reaction products, and FIG. 9D is pancreatic cancer cells treated with CP-Dex-Fe₃O₄ and shows no reaction product.

In addition, peptide histochemical diagnosis of the present invention can be applied to different types of cancers, including: lung cancer and neuroblastoma, which all reveal specific binding activity to FITC (Fluorescein isothiocyanate)-targeted peptide SEQ ID NO: 1; however, FITC-targeted peptide can not bind to the control immortalized embryonic renal epithelia (FIG. 1B). FITC-targeted peptide SEQ ID NO: 2 can be used to bind to hepatoma and pancreatic cancer cells. These results show that the targeted peptide can bind to NPC and other cancer cells but not bind to untransformed cells.

Another aspect of the present invention, adding a large amount dosage of the targeted peptide SEQ ID NO: 1 (such as 1 mg/mL) to the NPC cells culture medium is found no any specific change of tumor cell morphology and behavior, even if the culture condition sustained to 10 days. Apparently, this finding indicate that the protein bound by the targeted peptide is not a receptor protein or the like thereof.

Furthermore, that using the targeted peptide SEQ ID NO: 1-linked dextran-coated iron oxide nanoparticle (P-Dex-Fe₃O₄) to bind the tumor surgical specimens in the present invention can be applied to clinical chemotherapy. The binding ability of the targeted peptide SEQ ID NO: 1 in the paraffin-embedded NPC or other cancer sections can be observed, because more the targeted peptide SEQ ID NO: 1 inserts into dextran coated iron oxide nanoparticles, it is anticipated that the binding capability is excellent. Therefore, incubation of the paraffin-embedded sections with the targeted peptide SEQ ID NO: 1-linked dextran-coated iron oxide nanoparticles (P-Dex-Fe₃O₄) and using Prussian blue reaction can observe the tumor cells. The results show that the Prussian blue reaction product can be easily identified in the tumor cells of the paraffin-embedded sections, regardless of whether it is a tumor nest or a single infiltrating tumor cell.

The targeted peptide-linked dextran-coated iron oxide nanoparticles of the present invention reveal that the targeted peptide can bind to all types of cancer cells, therefore, the targeted peptide-linked dextran-coated iron oxide nanoparticles can effectively bind to breast cancer or other cancer cell types. However, using the targeted peptide SEQ ID NO:1 linked with biotin-modified-peptide (B-m-P) in the paraffin-embedded breast cancer sections reveal very weak or no binding signal, even though the same targeted peptide SEQ ID NO:1 is used. Accordingly, peptide histochemical method of present invention is used to bind to breast cancer surgical specimens, a similar good result is obtained, and it does not only reveal breast cancer cells in surgical specimens but also in the metastatic axillary lymph nodes.

In another aspect of present invention, the other targeted peptide SEQ ID NO: 2 can be used to synthesize the targeted peptide SEQ ID NO: 2-linked dextran-coated iron oxide nanoparticles, and the good result in hepatoma or pancreatic cancer sections similar with SEQ ID NO: 1-linked dextran-coated iron oxide nanoparticles can also be observed when using the targeted peptide SEQ ID NO: 2-linked dextran-coated iron oxide nanoparticle.

In another aspect of present invention, the targeted peptide-linked dextran-coated iron oxide nanoparticles have been proven to bind to nasopharyngeal carcinoma, breast cancer, hepatoma and pancreatic cancer cells in vitro and in vivo. Furthermore, peptide-targeted chemotherapy shows a high efficacy with minimal adverse effect for treatment of undifferentiated NPC and breast cancer. these results suggest that the targeted peptide-linked dextran-coated iron oxide nanoparticles of present invention has a multifunctional potential for clinical application in localization of its targeted protein in surgical specimens, which can further be applied to verifying the possible effectiveness of chemotherapy to each patients.

Accordingly, the present invention provides a peptide histochemical diagnosis which using the targeted peptide-linked dextran-coated iron oxide nanoparticles binding to different types of tumor cells in surgical specimens, wherein the targeted peptides only have a short sequence of 12 peptides, and the targeted peptide is a small molecular compared to the molecular weight of an antibody. Therefore, after linking to the nanoparticle, the short sequence peptide still has a tiny volume, so the targeted peptide-linked dextran-coated iron oxide nanoparticles can be allowed to enter into the extracellular space. Moreover, in the present invention, a single dextran coated iron oxide nanoparticle can conjugate with many the short sequence peptides, which allows more binding ability for binding to cancer cells in surgical paraffin section of the same cancers.

What is claimed is:

1. A method for peptide histochemical diagnosis, comprising:
a. providing a paraffin section of formalin-fixed tumor specimen obtained from sectioning and deparaffinizing a tumor specimen embedded in paraffin block;
b. providing a targeted peptide-dextran coated iron oxide nanoparticle, the surface of the iron oxide nanoparticle is coated with dextran, a N-terminal of the targeted peptide is linked to the dextran; and
c. incubating the paraffin section of tumor specimen with the targeted peptide-dextran coated iron oxide nanoparticle, then staining with a reagent to reveal a specific color, wherein a tumor cell in the paraffin section of tumor specimen reveals the specific color.

2. The method of claim 1, wherein the targeted peptide-dextran coated iron oxide nanoparticle is composed by at least 10 targeted peptides linked to the dextran coated iron oxide nanoparticle.

3. The method of claim 1, wherein a C-terminal of the targeted peptide of the targeted peptide-dextran coated iron oxide nanoparticle binds to the tumor cell.

4. The method of claim 1, wherein the paraffin section of tumor specimen is embedded in paraffin wax after formalin-fixed and dehydration.

5. The method of claim 1, wherein the tumor specimen further processes a high-pressure treatment to obtain the paraffin section of tumor specimen after deparaffinzing.

6. The method of claim 1, wherein the reagent is Prussian blue reagent.

7. The method of claim 6, wherein the specific color of Prussian blue reagent reacting with the iron oxide nanoparticle of the targeted peptide-dextran coated iron oxide nanoparticle is blue.

8. The method of claim 1, wherein a normal cell in the paraffin section of tumor specimen is not stained.

9. The method of claim 1, wherein the tumor is nasopharyngeal carcinoma, breast cancer, hepatoma, pancreatic cancer, small cell lung cancer or neuroblastoma.

10. A use of the targeted peptide-dextran coated iron oxide nanoparticles used in manufacturing the medicine for detecting a tumor specimen, wherein the tumor specimen is a paraffin section of tumor specimen.

11. A method of a targeted peptide-dextran coated iron oxide nanoparticle for detecting the peptide binding protein in a paraffin section of tumor specimen.

12. The method of claim 10, wherein a normal cell in the paraffin section of tumor specimen is not stained.

13. The method of claim 10, wherein the paraffin section of tumor specimen reveals a color if a tumor cell in the paraffin section of tumor specimen bound by the targeted peptide-dextran coated iron oxide nanoparticle.


SEQUENCE LISTING (4 sequences)

SEQ ID NO: 1 - protein, 12 aa, Artificial Sequence
  synthetic peptide
RLLDTNRPLLPY

SEQ ID NO: 2 - protein, 12 aa, Artificial Sequence
  synthetic peptide
SFSIIHTPILPL

SEQ ID NO: 3 - protein, 12 aa, Artificial Sequence
  synthetic peptide
RLLDTNRPLGGG

SEQ ID NO: 4 - protein, 5 aa, Artificial Sequence
  synthetic peptide
TKSGG